(19) United States
(12) Patent Application Publication
(10) Pub. No.: US 2008/0194918 A1
(43) Pub. Date: Aug. 14, 2008

(54) VITAL SIGNS MONITOR WITH PATIENT ENTERTAINMENT CONSOLE

(76) Inventors: Robert S. Kulik, Tampa, FL (US); Heather T. Menshouse, Odessa, FL (US); Bruce A. Friedman, Tampa, FL (US)

Correspondence Address: ANDRUS, SCEALES, STARKE & SAWALL, LLP, 100 EAST WISCONSIN AVENUE, SUITE 1100, MILWAUKEE, WI, 53202, US

(21) Appl. No.: 11/673,243

(22) Filed: Feb. 9, 2007

(51) Int. Cl. A61B 5/00

(52) U.S. Cl. 600/300

(57) ABSTRACT

A system and device for monitoring at least one physiological parameter of a patient and selectively presenting entertainment media and at least one physiological parameter on a common display. The system and device comprises a patient monitor disposed to collect at least one patient physiological signal and processing at least one physiological signal to obtain at least one physiological parameter. A patient entertainment console with a processor programmed to receive at least one physiological parameter and programmed to control the patient entertainment console to selectively present entertainment media and at least one physiological parameter upon receiving a signal from an override means.

### FIELD OF THE INVENTION

The present disclosure relates to clinical care systems and, more specifically, to the monitoring of patient physiological parameters and the display of the monitored physiological parameters.

### BACKGROUND OF THE INVENTION

Healthcare providers are facing increasing pressure to provide quality patient comfort and care at a lower cost. The automation of many patient monitoring tasks has been one solution to meeting these pressures. An example of this automation is in the medical or surgical wards of a hospital where the taking of vital signs is a routine procedure typically performed by clinicians. However, these procedures can be made more efficient through the use of an automated vital signs monitor (VSM). After the initial cost of the VSM, the healthcare provider is enabled to more efficiently perform these routine tasks. Thus, the clinician's effort may be directed towards other important tasks.

The high initial cost of a traditional VSM limits the ability of a healthcare provider to fully make the transition to VSMs by providing a VSM for each patient. On average, most healthcare providers only have one VSM for every seven to ten hospital beds. Thus, the VSMs must be employed in a mobile fashion. Productivity problems arise in this situation, as the mobile VSMs may be misplaced or not readily available when needed, and the VSMs are more prone to damage as they are transported about the hospital.

The increased use of patient monitoring systems, as well as the increased prevalence of items in the hospital rooms directed towards patient comfort, leads to cluttering of the area around the patient. Often, a patient may be connected to multiple patient monitoring and patient care systems that result in a plurality of fluid and electrical connections running to and from the patient. Many rooms also include a computerized data console that provides electronic access to the patient's medical records. Furthermore, to improve the comfort of the patient and any visitors that the patient may have, hospital rooms are outfitted with entertainment devices such as TVs and radios, communication devices, such as telephones, and furniture to accommodate visitors. Many of these items either require power cords that must be plugged into a wall electrical supply, or take up space in the already limited area of the hospital room. All of this leads to a decrease in the access that a clinician has to a patient in the normal course of providing care and in the event that additional or emergency care must be rendered. This increases the risk that the patient may be inadvertently harmed, or medical equipment may be inadvertently disconnected or damaged.

Products are available that attempt to combine some of the pieces of equipment found in a hospital room into a single unit. One such of these devices is a patient entertainment console (PEC), such as the Patientline™ Terminal, available from Patientline™. The PEC combines patient entertainment, patient communication, patient services, and/or clinical data access in a single console. The PEC replaces any patient entertainment equipment that may be in the room by providing the patient with access to television, movies, music, and/or video games. The PEC may also provide the patient with communication means, such as the Internet, e-mail, and/or telephone service. The PEC may further provide the patient with other services, such as hospital evaluation surveys, hospital information and/or education materials, and may provide the patient with the ability to place requests, such as calling a nurse, requesting room maintenance, or placing a food order. Therefore, this system provides the patient with services that previously may have required multiple, separate pieces of equipment to provide the same services to the patient.

The PEC further includes the ability for a clinician to use the PEC as a data console, such that the clinician may access a variety of patient data stored on a hospital information system, thus eliminating the need for a separate data console and the network connection associated with this console. The PEC provides the clinician with access to the patient medical history and charting, electronic medical records, patient medication administration schedules, PACS access to digital x-rays, the entry of physician orders, access to medical library information, and the reporting and documentation of patient requests and clinician actions.

Systems similar to the Patientline™ PEC have helped eliminate some of the peripheral devices that are commonly present in hospital rooms as individual pieces of equipment. However, the Patientline PEC is limited in that it only replaces the functionality of peripheral pieces of equipment and doesn't serve to help reduce the clutter of connections running to the patient and the pieces of equipment to which these patient connections must be connected.

Therefore, a system that further promotes hospital room efficiency by providing more functionality with fewer consoles and by providing a central location for receiving patient physiological data from patient connections is desirable in the field of hospital patient care.

### BRIEF DESCRIPTION

A patient monitoring device and system that collects patient physiological parameter data is herein disclosed. In ONE embodiment, the patient monitoring device comprises a plurality of patient connections that are disposed for connection to the patient and further comprises a data connection that is disposed to be connected to a display, which in an embodiment of the present invention is a patient entertainment console (PEC).

In a further embodiment a patient monitoring device comprises a PEC that displays entertainment media to a patient, and further comprises a patient monitor to which a plurality of patient connections are connected, such that the patient monitor collects physiological data from a patient.

In a still further embodiment, the patient monitor continuously collects patient physiological data, and in the embodiment, a clinician may select the data that is to be displayed by the PEC, wherein the data is either patient entertainment media data or the collected patient physiological data.

In another embodiment, the PEC comprises an override means whereby when the means is in a first, normally operating condition, the PEC displays patient entertainment media data and when the override means is activated by a clinician to a second condition, the PEC displays the patient physiological data as it is collected by the patient monitor.

In an additional embodiment, the PEC and a patient monitoring device are combined into a single device that is able to be manufactured and sold for less than two separate devices with equivalent functionality.

### BRIEF DESCRIPTION OF THE DRAWINGS

In the drawings:

FIG. 1 is a depiction of a first embodiment of the combined system;

FIG. 2 is a depiction of a second embodiment of the system;

FIG. 3 is a schematic block diagram of the combined patent monitoring device and the patient entertainment console of FIG. 1; and

FIG. 4 is a schematic block diagram of the integrated patient monitoring and entertainment console of FIG. 2.

### DETAILED DESCRIPTION

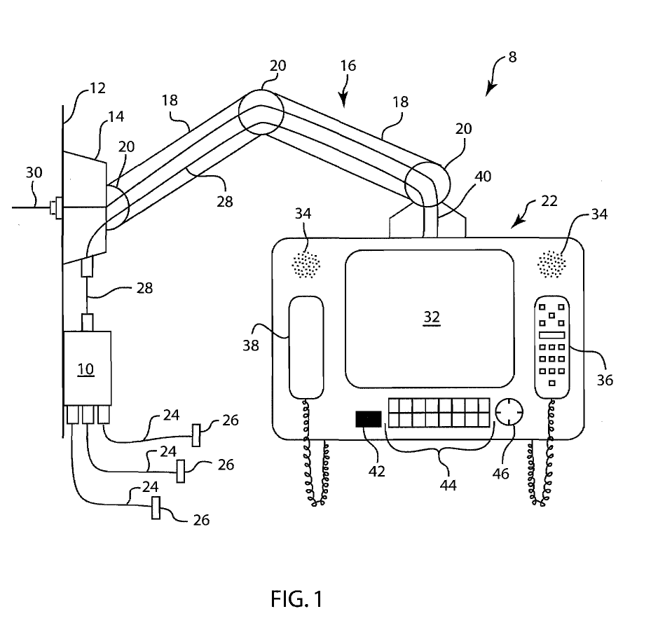

FIG. 1 depicts an embodiment of a patient monitoring device 8. The patient monitoring device 8 of FIG. 1 includes a patient monitor 10, which may be a vital signs monitor (VSM), is located close to a patient (not depicted). In the embodiment shown, the patient monitor 10 is secured to a wall 12 in close proximity to the patient and also in close proximity to a hanging bracket 14 also secured to the wall 12. Alternatively, the patient monitor may be secured to a pole stand (not depicted) in close proximity to the patient's bed. The hanging bracket 14 includes an articulated arm 16 that comprises limbs 18 and joints 20. The distal end of the articulated arm 16 supports a patient entertainment console (PEC) 22. In an alternative embodiment, the patient monitor 10 is secured to the patient's bed. The communications link 28 transmits data between the VSM and PEC and may comprise wired or wireless technology. Alternatively, the patient monitor 10 is located such that any connections between the patient, the patient monitor 10, and the PEC 22 are out of the way of the patient or a clinician attending to the patient.

The patient monitor 10 receives at least one patient connection 24. The patient connections 24 may end in an electrode 26 disposed to be removably attached to the skin of the patient and collect physiological data. Alternatively, the patient connections 24 may connect to other types of sensors or patient monitoring device (not depicted). The patient monitor 10 may comprise circuitry (not depicted) for the monitoring of a variety of patient physiological parameters, such as, but not limited to, blood pressure, pulse oximetry (SpO₂), temperature, heart rate, respiratory rate, electrocardiogram, or fluid input/output; however, this list is merely exemplary and is not intended to be limiting on the scope of the physiological parameters that may be monitored by the patient monitoring device 8. As can be seen in the embodiment of FIG. 1, this patient monitor 10 does not include a display device.

Once the physiological parameters of the patient are detected and collected by the patient monitor 10, the physiological data is sent to the patient entertainment console 22 via the communication link 28. In the embodiment shown in FIG. 1, the communication link 28 extends through the limbs 18 and joints 20 of the articulated arm 16 to the patient entertainment console 22. In an alternative embodiment the physiological data may be transmitted to the patient entertainment console 22 via a wireless connection (not depicted). In a still further embodiment (not depicted), the patient monitor 10 is connected to a hospital network 30 by the link 28 or another connection such that the physiological data received from the patient may be stored on a centralized server (not depicted) of the hospital network 30.

The patient entertainment console 22 comprises a display 32 and speakers 34. The patient entertainment console 22 can use the display 32 and the speakers 34 to present entertainment media data to the patient upon the selection of the entertainment by the patient or clinician. The patient may make this selection by the use of a remote control 36 that may be used to navigate through entertainment media selection and menu screens displayed on display 32. Alternatively, embodiments may utilize any alternative suitable data input device instead of a remote control 36, such as a touch screen or keyboard. The entertainment media data provided by display 32 and/or speakers 34 may include entertainment media data such as television programming, music, movies, and/or video games. The patient entertainment console 22 may also be used to provide communication functions to the patient. The communication functions may comprise a telephone handset 38. An Internet connection may be provided to the patient entertainment console 22 via a network connection 40 connecting the patient entertainment console 22 to the hospital network 30. With an Internet connection, the patient entertainment console 22 may provide the patient with access to the World-Wide Web or to internet-based e-mail communication.

The patient entertainment console 22 may also provide the patient with additional patient services such as access to hospital surveys, hospital information, medical education resources, or facilitate the patient's ability to make requests, such as calling a nurse, requesting room maintenance, or placing a food request.

The patient entertainment console 22 may further comprise an override means 42, such as a push button or toggle switch. The override means 42 controls the function of the patient entertainment console 22 such that when the override means 42 is in a first, normal condition, the patient entertainment console 22 provides the patient with access to the entertainment, communication, and service features that have presently been herein described. When the override means 42 is in a second, active condition, the patient medical data from the patient monitor 10 as the hospital information system is displayed to a nurse or clinician by the patient entertainment console 22, and the entertainment media data presentation features are deactivated. The override means 42 may be, but need not be, a physical button and/or switch. Alternatively the override means 42 may be any type of input device that is capable of performing a switching function, such as, but not limited to, a digital or graphical user interface (GUI) button or switch which may be activated by touch screen or cursor technology, a bar code scanner with which the clinician would scan a bar code to activate the override means 42, an electromagnetic strip reader, such that the clinician could swipe the electromagnetic strip of a hospital ID badge to activate the override means 42, or an RFID tag that is associated with the nurse or clinician and activates the override means 42 upon the clinician entering a close proximity of the patient entertainment console 22.

Once the override means 42 has been activated, the clinician has access to a variety of patient data, as would be provided to the clinician via either a separate data console (not depicted) or typical patient monitor display. The clinician may have access to the patient's medical history and/or charting data, electronic medical records, patient medication administration schedules, access to the hospital PACS system, such as digital X-rays, physician order entry, access to medical library resources, and the documentation and reporting of patient requests and clinician actions.

The clinician can interact with these functions of the patient entertainment console 22 and modify data within these functions via an input device, such as a series of buttons 44 or a data knob 46. However, many other types of input devices may be used by a clinician to navigate, enter, and modify patient data, such as the remote control 36, or other input means that are not depicted, such as a track ball, a touch screen, a keyboard, a mouse, a directional pad, or a bar code scanner.

The clinician may manipulate one of the input devices to activate the display 32 and to display the patient physiological data collected by the patient monitor 10 and sent to the PEC 22 via the communication link 28. Upon the clinician selection to display the monitored patient physiological parameters using the one of input devices, the PEC 22 will display the patient physiological parameters on the display 32 as they are collected by the patient monitor 10. The display 32 may be fully comprised by the patient physiological parameters, or alternatively, a portion of the display 32 may be dedicated to the display of the patient physiological parameters, while the remaining portion of the display may be used to display other patient data, as described previously. Therefore, the clinician would be able to observe the currently monitored patient physiological parameters and adjust or add to any patient data files to include the new patient physiological data.

In an embodiment, the patient monitor 10 monitors the patent physiological signals from the electrodes 26 connected to the patient. The patient monitor 10 sends the monitored patient physiological signals, via the hospital network 30, to a centralized patient data storage, such as a server, where the patient physiological parameters are recorded. In this embodiment, when the clinician activates the override means 42 to access the patient data, the clinician may access the past recorded patient physiological parameter data. Additionally, the clinician may manipulate the input device to initiate the display of the patient physiological data that is currently being monitored. This embodiment would allow the clinician to compare previous measurements of patient physiological parameters since the last time that the clinician had checked the patient's vital signs. Alternatively, the clinician may have access to the recorded patient physiological parameters from any computer workstation permitting the clinician access to the patient's medical data records, allowing the remote monitoring of the patient's condition.

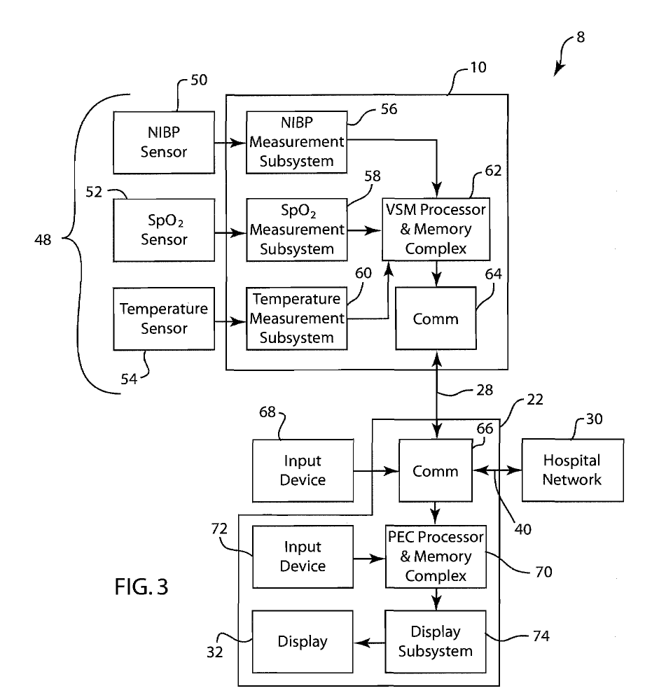

FIG. 3 depicts a schematic diagram of the functional electrical components required to implement the embodiment depicted in FIG. 1. It is understood that embodiments of the present invention may perform one or more of the functional components depicted in the schematic diagram of FIG. 3 with one or more physical electronic components, such as one or more microprocessors programmed to operate with the described functionality. The use of such electronic components as needed is within the scope of the present invention.

A plurality of sensors 48 are connected to the patient monitor 10. The plurality of sensors 48 may comprise a noninvasive blood pressure NIBP) sensor 50, an SpO₂ sensor 52, and a temperature sensor 54. These sensors 48 may also comprise electrodes 26 or may comprise other types of sensors commonly used to obtain patient physiological measurements, such as thermistors, pressure sensors, or optical sensors, all of which are merely exemplary of the types of sensors that may be used with the present invention and the listing herein is not intended to be limiting upon the types of sensors that may be used with the present invention. The sensors 48 transmit the physiological signals to the associated measurement subsystem of the patient monitor 10. Such measurement subsystems may comprise an NIBP measurement subsystem 56, an SpO₂ measurement subsystem 58, and a temperature measurement subsystem 60. The measurement subsystems collect the physiological signals from the sensors 48 and transmit the measured physiological signals to a vital sign monitor (VSM) processor and memory complex 62. The VSM processor and memory complex 62 receives the physiological signals from the measurement subsystems and processes the signals to obtain patient physiological data. This data is sent to a communications device 64. The communications device 64 transmits the patient physiological data over a communications link 28 to a corresponding communications device 66 of a patient entertainment console 22. In one embodiment, the communications link 28 is a wire; however, other forms of communications links 28 may be used in accordance with the present invention such as, but not limited to, RF communication or optical communication. The communications device 66 may be a router or a hub that controls data transfer decisions. Alternatively, the communications devices 64 and 66 may be an ethernet jack and the communications link 28 would be an ethernet cable extending between the communications devices 64 and 66.

The communications device 66 of the patient entertainment console 22 may also be disposed to receive data from other input devices 68 that are external to the patient entertainment console 22. These external input devices may transmit data to the communications device 66 via wire, RF or optical communications, and may include such input devices as a handheld barcode scanner or an RFID tag. The communications device 66 sends any patient physiological data received from the patent monitor 10 and any input data received from input devices 68 to the PEC processor and memory complex 70. The PEC processor and memory complex also receives input data from input devices 72 that are integral with the PEC 22. The input device 72, as previously described, may comprise keys or buttons, a remote control, a data knob, a trackball, a directional pad, or a barcode scanner.

The PEC processor and memory complex 70 facilitates the patient's selection of entertainment media data and the presentation of the selected entertainment media data by the PEC 22.

Upon receiving an input command received from the clinician, via either the external input devices 68 or the internal input devices 72, the PEC processor and memory complex 70 will control the display subsystem 24 and the display 32 to display the requested patient data to the clinician. Furthermore, upon the activation of the override means 42 (FIG. 1), which may comprise the external input devices 68 or the internal input devices 72, the PEC processor and memory complex 70 will control the display subsystem 74 and the display 32 to display patient data to the clinician. Further signals initiated by the clinician by activating an external input device 68 or an internal input device 72 direct the display 32 to display the measured patient physiological data on the display 32.

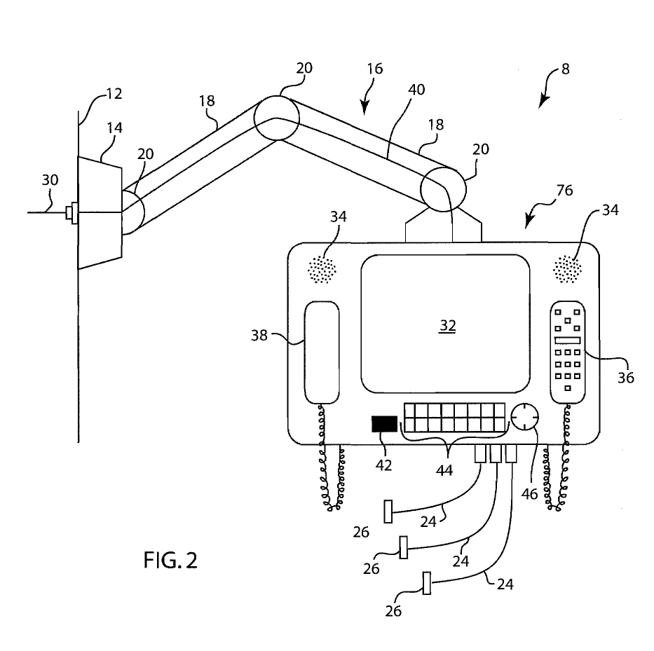

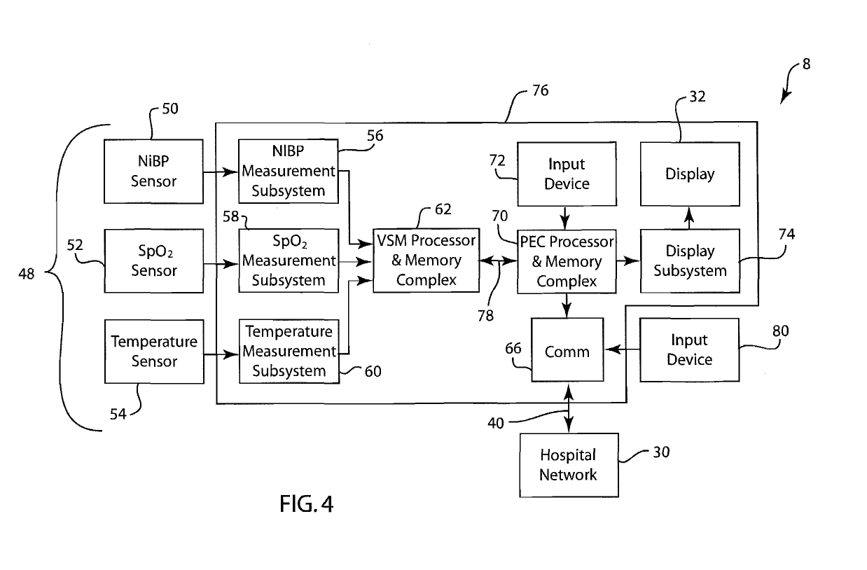

FIG. 2 depicts an alternative embodiment of the patient monitoring system 8. In this embodiment of the patient monitoring system 8, a single console 76 comprises the functionality of the patient entertainment console 22 and the monitoring device 10 of the embodiment depicted in FIGS. 1 and 3 into a single console 76. The internal operation of the console 76 will be described in further detail in regards to FIG. 4. In this embodiment of the present invention, the combined patient entertainment and patient monitoring console 76 may be connected to a wall 12 with a bracket 14 and an articulated arm 16 comprising limbs 18 and joints 20, although it is understood that embodiments of the console 76 may be located on a stand (not depicted) or mounted directly to a wall (not depicted).

The combined patient entertainment and monitoring console 76 includes a display 32 and a telephone 38 for providing the patient with telephonic communication. The console 76 may further comprise a variety of input devices as described in relation to the previously described embodiments such as, but not limited to, a remote control 36, a data knob 46, and buttons or keys 44. Furthermore, the console 76 may comprise an override means 42 such that the clinician may switch the console from displaying the patient entertainment media data to the patient to displaying the patient medical data to the clinician.

In the embodiment of FIG. 2, no external patient monitor 10 is present as in the embodiment of the patient monitoring system 8 shown in FIG. 1. Rather, the functional components of the patient monitor 10 are integrally located within the combined patient entertainment and monitoring console 76. The patient connections 24 extend from the console 76 to the electrode or other sensors 26 that are attached to the patient (not depicted) or to other patient monitoring devices (not depicted). Furthermore, because the monitoring device 10 is not an external component to the patient entertainment console 22, no external communications link 28 is necessary to transmit the patient physiological data from the monitoring device 10 to the patient entertainment console 22, as this connection is an internal connection 78 (FIG. 4) within the combined patient entertainment and monitoring console 76.

Referring now to FIG. 4, a schematic diagram of the functional components of the patient monitoring system of FIG. 2 is depicted. It is understood that one or more of the functional components depicted in the schematic diagram of FIG. 4 may be practiced by one or more electronic components, such as a microprocessor or CPU within the patient entertainment and monitoring console 76. The patient entertainment and monitoring console 76 comprises a VSM processor and memory complex 62 and a PEC processor and memory complex 70. The VSM processor and memory complex 62 receives patient physiological signals that are collected from a plurality of sensors 48 such as a noninvasive blood pressure (NIBP) sensor 50, an SpO₂ sensor 52, and a temperature sensor 54. The sensed patient physiological signals are sent to a respective measurement subsystem such as a noninvasive blood pressure measurement subsystem 56, an SpO₂ measurement subsystem 58, and a temperature measurement subsystem 60, where the raw patient physiological signals are processed to be sent to the VSM processor and memory complex 62. The measurement subsystems may comprise electronic components such as amplifiers, filters and/or an analog-to-digital converter. The processed physiological signals are sent from the measurement subsystems to the VSM processor and memory complex 62 where the physiological signals are processed into the desired patient physiological data. This patient physiological data is sent to the PEC processor and memory complex 70 via an internal connection 78.

The PEC processor and memory complex 70 perform the general processing duties and control for the combined patient entertaimnent and monitoring console 76. In the patient entertainment mode, the PEC processor and memory complex 70 receives input data from a plurality of input devices 80, which may comprise any of the input devices 80 for the patient entertainment console as have been herein described. The input data is indicative of a patient's selection of entertainment media data to be presented to the patient. Upon selection of the entertainment media data, the PEC processor and memory complex 70 will retrieve the selected entertainment data either from the memory of the PEC processor and memory complex 70 or via a data connection 40 connecting the console 76 to the hospital network 30 and any centralized storage data on the hospital network, or any data transmission, such as television available through the hospital network 30. The PEC processor and memory complex 70 controls the display subsystem 74 to operate the display 32 to present the selected entertainment media data.

Upon the initiation of the override means, which may comprise one of the input devices 80 the clinician can control; the PEC processor and memory complex 70 discontinues presenting the patient entertainment media data and instead retrieves the patient medical data from the hospital network 30 via the network connection 40 to the communications transceiver 66. Upon another input from the clinician operating one of the input devices 80, the PEC processor and memory complex 70 controls the display subsystem 74 and display 32 to display the patient physiological data that is processed by the VSM processor and memory complex 62. The VSM processor and memory complex 62 transmits the patient physiological data to the PEC processor and memory complex 70 to be displayed on the display 32. Alternatively, the console 76 does not actively monitor the patient physiological signals and may require the clinician to activate a signal with an input device 80 to initiate the monitoring of the patient physiological signals. The signal from the input device 80 is received by the PEC processor and memory complex 70, which in turn sends a control signal to the VSM processor and memory complex 62. Therefore, the patient's vital signs are selectively monitored only upon the VSM processor and memory complex 62 receiving a control signal from the PEC processor and memory complex 70.

The following is a description of an exemplary interaction between a patient and a clinician utilizing an embodiment patient monitoring system as depicted in FIG. 2 to more fully explain aspects of an embodiment of the present invention. The patient may use the remote 36 to control the patient entertainment media data that is presented to the patient via the display 32 and/or the speakers 34 of the patient entertainment and monitoring console 76. Upon the selection of entertainment media data, the console 76 acquires this data, either from the memory (not depicted) of the console 76, or via the network connection 40 connecting the console 76 to the data available on the hospital's network 30, which may comprise a variety of patient entertainment media data.

When the clinician enters the patient's room, the clinician invokes an override function of the console 76 by an override means 42. The override function suspends the presentation of the entertainment media data by the console 76 and switches to the display of patient medical data. Alternatively, the override function may direct the display 32 to display a generalized patient data screen whereby the clinician is required to enter either clinician identification information, patient identification information or both into the console 76 in order to access the patient medical history data. The clinician may do this by using a barcode scanner to scan a barcode located on the patient's ID wristband, swiping a electronic strip of an ID badge, using an RFID tag, or entering a patient ID number into the console 76 via a keyboard or other input device.

The console 76 then displays the patient's medical data to the clinician. Next the clinician activates the monitoring of patient physiological parameters. In an embodiment, the vital signs sensors 48 have already been placed on the patient's body in the appropriate location, and the console 76 has been monitoring the patient's physiological parameters. In this embodiment, the console simply switches to display the monitored patient data on the display 32. In an alternative embodiment of the present invention, no monitoring of the patient's vital sign may have been performed up to this point, and as such, the clinician must place vital sign sensors 48 on the patient's body and connect the sensors 48 to the console 76. The clinician then initiates the monitoring of the patient's physiological parameters, whereby the console 76 monitors the patient's physiological parameters and displays these parameters on the display 32. The clinician can read and interpret the patient's physiological parameter data that is displayed on the display 32, and the physiological parameter data may be saved to the patient's medical data. After the clinician has checked the patient's physiological parameters, the clinician may once again use the override means 42 of the console 76 to return the console 76 to providing the patient with a presentation of entertainment media data.

In an alternative embodiment, the clinician may override the display of entertainment media data on the console 76 using the override means 42. Once this has been accomplished, the clinician may use the input device 80 to control the VSM processor 62 to collect physiologic data at pre-determined intervals. The pre-determined intervals may comprise specific intervals and data collection durations for each of the monitored physiological parameters. The clinician may then switch the console 76 back to displaying entertainment media data using the override means 42. Thus, while the entertainment media data is being displayed by the console 76, the VSM processor 62 is collecting the patient physiologic data at the pre-determined intervals. The physiologic data may be buffered or conveyed through the hospital network for storage until the clinician again overrides the console 76 display to access the patient data.

The combined system described exhibits many advantages over other presently available patient monitoring and patient entertainment devices. The patient monitoring device provides a cost savings because no separate display is required for the patient monitoring device, rather the patient monitoring device uses the display that is already required for the patient entertainment console, thus reducing the costs for the combined units. Furthermore, the display for the patient entertainment console is typically a higher quality display than displays normally provided with patient monitoring systems. Therefore, the patient monitoring device provides the additional benefit of utilizing a higher quality display for the display of patient physiological data. In an embodiment of the present invention wherein the patient monitoring device is separate from the patient entertainment console, a further advantage is found in that a hospital that already owns a patient entertainment console can add the functionality of a patient monitoring device for a relatively low cost, as compared to a full patient monitoring device. In the embodiment utilizing a combined patient entertainment and monitoring console, a further reduction in the cost of providing both the patient monitoring device and the patient entertainment console in each hospital room is further reduced because both devices are found in one unit, this reduces manufacturing costs as well as allows for the elimination of duplicate components such as the device housing, power supply, and communication transceivers.

An additional advantage is that the clutter in the hospital room is reduced by the elimination of a stand-alone patient monitoring device. In the embodiment comprising a combined patient entertainment and monitoring console, this advantage is further increased because the embodiment allows for shorter patient connections. The patient connections extend from the sensors to the console. Typically, the console is placed at a location close to the patient such that the patient can utilize the input function of the console. Therefore, the length of the patient connections may be reduced. Furthermore, the console is typically connected to an articulated arm that is secured to a wall of the hospital room. This allows the console to be moved away from the patient in the event that the clinician needs greater access to the patient, such as to provide emergency care. The reduction in the patient connection length prevents the patient connections from coming in contact with the floor, where the patient connections may become a trip hazard to clinician or visitor for the patient, or the patient connection may potentially pick up dangerous pathogens. Additionally, an advantage of the system is that the noninvasive blood pressure monitoring functions of the patient monitor are improved because the shorter patient connections leads to faster and more accurate readings of patient blood pressure. A further advantage is that the VSM is permanently located in the patient room, eliminating the need to search for a VSM when needed and reducing the amount of equipment that must be transported by the caregiver into the patient care area.

This written description uses examples to disclose features of the embodiments, including the best mode, and also to enable any person skilled in the art to make and use the invention. The patentable scope is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

Various alternatives and embodiments are contemplated as being with in the scope of the following claims, particularly pointing out and distinctly claiming the subject matter regarded as the invention.

1-14. (canceled)

15. The patient entertainment and monitoring device of claim 27, further comprising an articulated arm extending between a housing, within which at least the VSM processor and the PEC processor are disposed, and a bracket, the bracket being securely fastened to a wall.

16. The patient entertainment and monitoring device of claim 27, wherein the physiological data comprises at least non-invasive blood pressure data, SpO₂ data, and temperature data.

17-26. (canceled)

27. A patient entertainment and monitoring device, the device comprising:
a graphical display that graphically presents data;
patient connection that is removably attached to a patient to collect physiological signals from the patient;
a VSM processor that receives the physiological signals from the patient connection and processes the physiological signals to produce physiological data;
an entertainment data source that comprises entertainment data;
a PEC processor connected to the VSM processor, the entertainment data source, and the graphical display, the PEC processor operating in a first condition wherein the PEC processor causes the entertainment data from the entertainment data source to be presented by the graphical display and operating in a second condition wherein the PEC processor causes the physiological data from the VSM processor to be presented by the graphical display; and
an RFID sensor connected to the PEC processor that receives an RFID signal from an RFID tag in close proximity with the RFID sensor wherein when the RFID sensor receives an RFID signal, the PEC processor switches from the first condition to the second condition.

28. The patient entertainment and monitoring device of claim 27, wherein when the PEC processor is operating in the second condition the presentation of the entertainment data on the graphical display is disabled.

29. The patient entertainment and monitoring device of claim 28, further comprising an input device connected to the PEC processor, the input device receiving modifications to the presented physiological data when the PEC processor is in the second condition.

30. The patient entertainment and monitoring device of claim 27, wherein the entertainment data source is an entertainment database comprising entertainment data, the entertainment database being connected to the PEC processor.

31. The patient entertainment and monitoring device of claim 30, further comprising a first database connected to the PEC processor, the first database storing patient data, wherein when the PEC processor operates in the second condition, the patient data is presented by the graphical display.

32. The patient entertainment and monitoring device of claim 31, further comprising an input device connected to the PEC processor, the input device receiving modifications to the presented patient data when the PEC processor is in the second condition.

33. A method for presenting entertainment data and physiological data using a patient entertainment console including a graphical display, a patient monitoring device, a processor, and an RF sensor, the method comprising the steps of:
acquiring a physiological signal from a patient with the patient monitoring device;
processing the physiological signal with the patient monitoring device to produce physiological data;
presenting entertainment data to the patient with the graphical display by operating the processor in a first condition;
receiving an RF signal with the RF sensor, the RF signal emanating from an RFID tag associated with a clinician; and
upon receiving the RF signal, disabling the presentation of entertainment data and presenting physiological data with the graphical display by operating the processor in a second condition.

34. The method of claim 33, further comprising the steps of:
upon the termination of the receipt of the RF signal by the RF sensor, discontinuing the presentation of physiological data on the graphical display; and
presenting the entertainment data on the graphical display.

35. A method for presenting entertainment data and physiological data using a patient entertainment console connected to an information network, the method comprising the steps of:
acquiring entertainment data from an entertainment data source connected to the information network;
presenting the entertainment data on a graphical display of the patient entertainment console;
acquiring physiological data from a patient connected to a patient monitoring device of the patient entertainment console;
receiving an RF signal with an RF sensor of the patient entertainment console, the RF signal being produced by an RFID tag associated with a clinician in close proximity to the patient entertainment console;
upon receiving the RF signal, disabling the presentation of the entertainment data on the graphical display; and
presenting the physiological data on the graphical display.

36. The method of claim 35 further comprising the steps of:
upon the termination of the receipt of the RF signal by the RF sensor, discontinuing the presentation of physiological data on the graphical display; and
presenting the entertainment data on the graphical display.

37. The method of claim 36 wherein the RF sensor receives the RF signal only when the RFID tag is in a close proximity to the patient entertainment console.

38. The method of claim 35 further comprising the steps of:
acquiring patient data from a patient data source connected to the information network; and
upon receiving the RF signal, presenting the patient data on the graphical display.

39. The method of claim 38 further comprising the steps of:
receiving, a clinician modification to the patient data;
saving the clinician modification to the patient data on the patient data source.
